(19) United States
(12) Patent Application Publication
(10) Pub. No.: US 2013/0323810 A1
(43) Pub. Date: Dec. 5, 2013

(54) BIOLOGICAL METHOD FOR LIQUID FERRIC SULFATE MANUFACTURING

(71) Applicant: BACTECH ENVIRONMENTAL CORPORATION, Toronto (CA)

(72) Inventors: Murray Ross ORR, Toronto (CA); Lou NAGY, Newmarket (CA); Paul Charles MILLER, Ocean Reef (AU); Junxiang GUO, Mississauga (CA)

(73) Assignee: BACTECH ENVIRONMENTAL CORPORATION, Toronto (CA)

(21) Appl. No.: 13/830,440

(22) Filed: Mar. 14, 2013

Related U.S. Application Data
(60) Provisional application No. 61/655,283, filed on Jun. 4, 2012.

(51) Int. Cl. C12P 3/00

(52) U.S. Cl. CPC C12P 3/00
USPC 435/168

(57) ABSTRACT

The present document describes a process for manufacturing liquid ferric sulfate from an iron sulfide material provided from ore tailings or mine tailings, the process comprising the step of contacting an aqueous solution containing the iron sulfide material with a thermotolerant bacteria culture capable of promoting oxidation of the iron sulfide material for producing liquid ferric sulfate effected without a sulfuric acid addition.

### CROSS-REFERENCE TO RELATED APPLICATIONS

This application claims priority under 35USC§119(e) of U.S. provisional patent application 61/655,283, filed on Jun. 4, 2012, the specification of which is hereby incorporated by reference.

### BACKGROUND

(a) Field

The subject matter disclosed generally relates to a bio-oxidation process for the manufacture of liquid ferric sulfate. More particularly, the subject matter disclosed relates to a bio-oxidation process for the manufacture of liquid ferric sulfate from an iron sulfide.

(b) Related Prior Art

Tailings are the materials left over after the process of separating the valuable fraction from the uneconomic fraction of an ore. Tailings are distinct from overburden, which is the waste rock or materials overlying an ore or mineral body that are displaced during mining without being processed.

The extraction of minerals from ore can be done two ways: placer mining, which uses water and gravity to extract the valuable minerals, or hard rock mining, which uses pulverization of rock, then chemicals. In the latter, the extraction of minerals from ore requires that the ore be ground into fine particles, so tailings are typically small and range from the size of a grain of sand to a few micrometers. Mine tailings are usually produced from the mill in slurry form, i.e., a mixture of fine mineral particles and water.

Tailings represent an external cost of mining, particularly true of early mining operations which did not take adequate steps to make tailings areas environmentally safe after closure. Modern day mines, particularly in jurisdictions with well-developed mining regulations or operated by responsible mining companies, incorporate the rehabilitation and proper closure of tailings areas in the mining costs and activities. For example, the province of Quebec, Canada, requires not only submission of closure plan before the start of mining activity, but also the deposit of a financial guarantee equal to 100% of the estimated rehabilitation costs. Tailings dams are often the most significant environmental liability for a mining project. There is therefore a need to exploit the tailing areas in the mining industry.

With the formula FeS₂, pyrite is the most common sulfide mineral and is widely associated with other metal sulfide deposits. Unless it contains valuable metals to be recovered, pyrite is usually rejected into tailings by beneficiation processes. Such tailings represent a large disposal problem because pyrite gets oxidized and generates sulfuric acid after being exposed to air and water. Pyrite oxidation is by far the greatest contributor of acid mine drainage and/or acid rock drainage.

For example, ferric sulfate is a staple chemical with a wide range of applications. It is commonly used as a coagulant in potable and wastewater clarification including turbidity removal, color removal, phosphate removal, heavy metal removal, in lime softening applications as well as for sludge conditioning. In the mining industry, ferric sulfate is not only a leaching lixiviant in various processes treating copper concentrates and uranium ores, but also a reagent commonly used to control arsenic in metal mining effluents. Ferric sulfate may be manufactured using a plurality of different processes.

As for example, U.S. Pat. No. 4,814,158 (Mar. 21, 1989) describes a process for the production of liquid ferric sulfate from finely-divided ferric oxide, sulfuric acid and water in a closed reaction vessel at temperatures ranging from about 130° C. to about 150° C., and pressures from about 30 psi to about 40 psi. The reaction time ranges from four to eight hours and produces liquid ferric sulfate having at least 10% trivalent iron. This process involves a high range of reaction temperature and a high range of reaction pressure.

As for another example, U.S. Pat. No. 6,375,919 (Apr. 23, 2002) describe a method for the manufacture of a ferric sulfate solution, characterized in that, iron ore containing 30% by weight or more FeOOH as a trivalent iron is calcined at 200-600° C., and then dissolved in sulfuric acid. This process also involves a high range of reaction temperature and a high range of reaction pressure.

Moreover, U.S. Pat. No. 7,387,770 (Jun. 17, 2008) describes a process for the production of liquid ferric sulfate from finely-divided ferric oxide, sulfuric acid and water in a reaction system comprising at least one closed reaction vessel at temperatures ranging from about 120° C. to about 150° C. and pressures from about 25 psi to about 70 psi. As mentioned above, this process involves a high range of reaction temperature and a high range of reaction pressure.

The art of bioleaching or bio-oxidation has been well developed since the late 1980's. One of the technologies developed relates to a process for recovering base and/or precious metals from sulfide materials comprising bacterial leaching the sulfide material with an aqueous solution at a temperature of 25° C. to 55° C. The aqueous solution contains a thermotolerant bacterial culture which has an optimum growth temperature of 40° C. to 45° C., and is capable of promoting oxidation at a temperature of 25° C. to 55° C. The oxidized residue is separated from the aqueous liquid and metal is recovered from the residue and/or aqueous solution.

Another one of the technologies developed relates to a process for recovering precious metals from particulate sulfide materials comprising bacterial leaching the sulfide material with an aqueous solution at a temperature of 45° C. to 90° C. The aqueous solution contains a facultative or obligate thermopile capable or promoting the oxidation of the sulfide material. The oxidized residue is separated from the residue and/or aqueous solution. The bioleaching is carried out at a temperature of at least 45° C., and in the presence of nutrients to oxidize the mineral sulfide and liberate the precious metal. This technology relates mainly to the use of higher temperature bacteria which is more relevant for base metal extraction.

Consider a large number of end users of ferric sulfate are operating mines and mining communities in remote areas, and pyrite is ubiquitous in those areas where sulfide ores are being mined, it is envisaged that modular bioleaching plants be installed to manufacture liquid ferric sulfate.

There is therefore a need for an improved process for manufacturing liquid ferric sulfate.

### SUMMARY

It is an object of the present invention to provide a bacterial oxidation process for utilizing profitability the most common of the iron sulfides.

According to an embodiment, there is provided a process for manufacturing liquid ferric sulfate from an iron sulfide material provided from ore tailings or mine tailings, the process comprising the step of contacting an aqueous solution containing the iron sulfide material with a thermotolerant bacteria culture capable of promoting oxidation of the iron sulfide material for producing liquid ferric sulfate effected without a sulfuric acid addition.

The iron sulfide material may comprise one of: pyrite, pyrrhotite, troilite, greigite, magnetite, mackinawite, marcasite and any combination thereof.

Contacting the aqueous solution containing the iron sulfide material with the thermotolerant bacteria culture may occur in one of: an agitated tank, an agitated reactor, a non-mechanically agitated column, a non-mechanically agitated tank, a non-mechanically agitated reactor, a pipe reactor, a reactor, a batch reactor, a continue reactor, a stacked heap, a dump leaching, a heap leaching, non-stacked piles, stacked piles, an aerated reactor, a non-aerated reactor and any combination thereof.

The thermotolerant bacteria culture may include an optimum growth temperature of 40 to 45° C.

Contacting the aqueous solution containing the iron sulfide material with the thermotolerant bacteria culture may occur at a temperature in the range from 25 to 55° C.

The thermotolerant bacteria culture may be capable of promoting oxidation of the iron sulfide material at a temperature in the range from 25 to 55° C.

Contacting the aqueous solution containing the iron sulfide material with the thermotolerant bacteria culture may comprise solubilizing the thermotolerant bacteria culture in the aqueous solution.

The process may further comprise the step of grounding or crushing the iron sulfide material prior to contacting the aqueous solution containing the iron sulfide material with the thermotolerant bacteria culture.

Contacting the aqueous solution containing the iron sulfide material with the thermotolerant bacteria culture may follow a chemical reaction defined by:

4FeS₂+15O₂+2H₂O→2Fe₂(SO₄)₃+2H₂SO₄ (i)

The chemical reaction may include individual reactions defined by:

2FeS₂+7.5O₂+H₂O→2FeSO₄+2H₂SO₄ (1)

4FeSO₄+O₂+2H₂SO₄→2Fe₂(SO₄)₃+2H₂O (2)

FeS₂+Fe₂(SO₄)₃→3FeSO₄+2S (3)

2S+3O₂+2H₂O→2H₂SO₄ (4)

Contacting the aqueous solution containing the iron sulfide material with the thermotolerant bacteria culture may occur at a pressure in the range from 10 psi to 20 psi.

Contacting the aqueous solution containing the iron sulfide material with the thermotolerant bacteria culture may occur at a pressure of 14.7 psi (1 atm).

The iron sulfide material may comprise an iron sulfide material concentrate.

According to another embodiment, in a process for producing liquid ferric sulfate, the improvement may consist in contacting an aqueous solution containing an iron sulfide material with a thermotolerant bacteria culture capable of promoting oxidation of the iron sulfide material for producing liquid ferric sulfate effected without a sulfuric acid addition.

The following terms are defined below.

The term “aqueous solution” is intended to mean a solution in which solvent is water as water is naturally abundant and may be found, without limitations, in rain, ocean, rivers, lakes, mining sites, and the like. It is understood by this definition that “aqueous solution” is not mixed with sulfuric acid, except in the case of acid rains, or the like.

Features and advantages of the subject matter hereof will become more apparent in light of the following detailed description of selected embodiments. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

### DETAILED DESCRIPTION

In embodiments, there are disclosed a bio-oxidation process for manufacturing liquid ferric sulfate from an iron sulfide. More particularly, in embodiments, there are disclosed a process for manufacturing liquid ferric sulfate from iron sulfide material provided from ore tailings or mine tailings.

According to an embodiment, there is described a process for manufacturing liquid ferric sulfate from an iron sulfide material provided from ore tailings or mine tailings. The process may include the step of contacting an aqueous solution containing the iron sulfide material with a thermotolerant bacteria culture capable of promoting oxidation of the iron sulfide material for producing liquid ferric sulfate. It is to be noted that contacting the aqueous solution containing the iron sulfide with the thermotolerant bacteria is effected without a sulfuric acid addition.

According to another embodiment, in the process, the iron sulfide material may include, without limitation, one of: pyrite, pyrrhotite, troilite, greigite, magnetite, mackinawite, marcasite and the like. It is to be mentioned that the iron sulfide material may include a source of iron carbonate such as, without limitations, siderite.

According to an embodiment, contacting the aqueous solution containing the iron sulfide material with a thermotolerant bacteria culture of the process may occur, without limitations, in one of: an agitated tank, an agitated reactor, a non-mechanically agitated column, a non-mechanically agitated tank, a non-mechanically agitated reactor, a pipe reactor, a reactor, a batch reactor, a continue reactor, a stacked heap, a dump leaching, a heap leaching, non-stacked piles, stacked piles, an aerated reactor, a non-aerated reactor and the like. It is to be noted that reactors mentioned above may be intentionally aerated or may not as the case may be.

According to another embodiment, the thermotolerant bacteria culture of the process may include an optimum growth temperature of 40 to 45° C. The step of contacting an aqueous solution containing the iron sulfide material with a thermotolerant bacteria culture may occur at a temperature in the range from 25 to 55° C.

According to another embodiment, the thermotolerant bacteria culture is capable of promoting oxidation of the iron sulfide material at a temperature in the range from 25 to 55° C.

According to another embodiment, the step of contacting the aqueous solution containing the iron sulfide material with the thermotolerant bacteria culture may be followed by the step of solubilizing the thermotolerant bacteria culture with the aqueous solution containing the iron sulfide material.

According to another embodiment, the process may further include the step of grounding or crushing the iron sulfide material prior to the step of aqueous solution containing the iron sulfide material.

According to an embodiment, the process may include the following overall chemical reaction:

4FeS₂ (pyrite)+15O₂+2H₂O→2Fe₂(SO₄)₃+2H₂SO₄ (i)

According to another embodiment, the overall chemical reaction (i) of the process may include the individual following reactions:

2FeS₂+7.5O₂+H₂O→2FeSO₄+2H₂SO₄ (1)

4FeSO₄+O₂+2H₂SO₄→2Fe₂(SO₄)₃+2H₂O (2)

FeS₂+Fe₂(SO₄)₃→3FeSO₄+2S (3)

2S+3O₂+2H₂O→2H₂SO₄ (4)

According to another embodiment, the step of the process 100 of contacting the aqueous solution containing the iron sulfide material may occur at a pressure in the range from 10 psi to 20 psi. More particularly, the step 102 of the process 100 of contacting the aqueous solution containing the iron sulfide material may occur at a pressure of 14.7 psi, i.e., 1 atm.

According to another embodiment, the iron sulfide material of the process may include an iron sulfide material concentrate.

The present invention will be more readily understood by referring to the following examples which are given to illustrate the invention rather than to limit its scope.

### Example 1

### Pyrite as the Iron Sulfide Material

Pyrite contains approximately 53% sulphur. The reactions for bacterial oxidation of pyrite given below indicate the role of both the direct and indirect mechanisms and how they are synergistic to promote leaching or bio-oxidation or bacterial oxidation.

### Overall Reaction:

$$\begin{matrix}4 Fe {S}_{2} + & 15 {O}_{2} + & 2 {H}_{2} O -> & 2 {{Fe}_{2} ( S {O}_{4} )}_{3} + & 2 {H}_{2} S {O}_{4} \\ ( pyrite ) & ( oxygen ) & ( water ) & ferric sulfate & sulfuric acid\end{matrix} \qquad ( i )$$

### Individual Reactions:

2FeS₂+7.5O₂+H₂O→2FeSO₄+2H₂SO₄ (1)

In the presence of bacteria, the ferrous sulphate will be re-oxidised to ferric sulphate, consuming oxygen and sulphuric acid according to:

4FeSO₄+O₂+2H₂SO₄→2Fe₂(SO₄)₃+2H₂O (2)

The chemical oxidation of pyrite by ferric sulphate, under the conditions normally encountered in a bacterial-oxidation reactor, proceeds as follows:

FeS₂+Fe₂(SO₄)₃→3FeSO₄+2S (3)

Elemental sulphur is oxidised in the presence of bacteria according to the following reaction, resulting in the formation of sulphuric acid:

2S+3O₂+2H₂O→2H₂SO₄ (4)

A comparison of reaction conditions shows distinct advantages of the process for manufacturing liquid ferric sulfate from an iron sulfide material over existing patented manufacturing processes in the market:

TABLE 1

| | | Reaction | Reaction |
|---|---|---|---|
| | Feed | Temp. | Pressure |
| Pat. | Material | (° C.) | (psi) |
| U.S. Pat. No. 4,814,158 | ferric oxide | 130-150 | 30-40 |
| U.S. Pat. No. 6,375,919 | ferric oxide | 200-600 | 15 |
| U.S. Pat. No. 7,387,770 | ferric oxide | 120-150 | 25-70 |
| Present | iron | 25-55 | 14.7 (= 1 atm) |
| process | sulfides | | |

In addition to the differences in reaction conditions, existing patented manufacturing processes require good quality iron oxide concentrates in order to minimize the consumption of concentrated sulphuric acid. Any manufacturing plant which uses concentrated sulphuric acid as a reactant has to be located strategically in areas where concentrated sulphuric acid is readily available.

TABLE 2

| | | | Concentrated | |
|---|---|---|---|---|
| | Feed Material | | Sulphuric Acid | |
| | | Total Fe (% | | Purity |
| Pat. | Type | wt.) | Usage | (%) |
| U.S. Pat. No. 4,814,158 | iron oxide | 98 | Yes | 93 |
| U.S. Pat. No. 6,375,919 | iron oxide | >30% | Yes | 30-50 |
| | | FeOOH | | |
| U.S. Pat. No. 7,387,770 | iron oxide | 93-98 | Yes | 90 |
| Present | iron | flexible | No | n/a |
| process | sulfides | | | |

The process is of great value in removing this constraint of the need for concentrated sulphuric acid and also creates greater flexibility for the type of feeds which could be utilized profitably. For example, a large number of end users of ferric sulfate are operating mines and mining communities in remote areas. As long as there is pyrite in the ore or mine tailings, liquid ferric sulfate can be manufactured by the present process.

As the most common of the sulfide minerals, pyrite in the ores and tailings can be enriched into very high purity concentrates by conventional froth flotation method widely used in the mining industry. For illustration purposes, it is assumed a pyrite feed that is 100% pure and the quality of liquid ferric sulfate is calculated, liquid ferric sulfate which would be produced in a batch mode at different solids concentrations by the present process 100 (see Table 3 and Table 4).

TABLE 3

| | | | | | | | Pulp | | | | |
|---|---|---|---|---|---|---|---|---|---|---|---|
| | | | Oxygen | | | | Weight | | | | |
| Pulp | Pyrite | | (O₂) | | Water | | at end | Free H₂SO₄ | | | |
| Density | (FeS₂) | | (from air) | | (H₂O) | | point | in final product | | | |
| (% solids) | (mol) | (g) | (mol) | (g) | (g) | (ml) | (g) | (mol) | (g) | (wt. %) | (g/L) |
| 10 | 1 | 120 | 3.75 | 120 | 1080 | 1080 | 1320 | 0.5 | 49 | 3.7 | 45 |
| 15 | 1 | 120 | 3.75 | 120 | 680 | 680 | 920 | 0.5 | 49 | 5.3 | 72 |
| 20 | 1 | 120 | 3.75 | 120 | 480 | 480 | 720 | 0.5 | 49 | 6.8 | 102 |
| 25 | 1 | 120 | 3.75 | 120 | 360 | 360 | 600 | 0.5 | 49 | 8.2 | 136 |

TABLE 4

| Pulp | [Fe³⁺] in | | | | [SO₄] in | | | | |
|---|---|---|---|---|---|---|---|---|---|
| Density | final product | | | | final product | | | | Molar |
| (% | | | (wt. | | | | (wt. | | Ratio |
| solids) | (mol) | (g) | %) | (g/L) | (mol) | (g) | %) | (g/L) | SO₄/Fe |
| 10 | 1 | 56 | 4.2 | 52 | 2 | 192 | 14.5 | 178 | 2 |
| 15 | 1 | 56 | 6.1 | 82 | 2 | 192 | 20.9 | 282 | 2 |
| 20 | 1 | 56 | 7.8 | 116 | 2 | 192 | 26.7 | 400 | 2 |
| 25 | 1 | 56 | 9.3 | 155 | 2 | 192 | 32.0 | 533 | 2 |

It is understood that the quality criteria of liquid ferric sulfate depend on its applications. Only the ferric sulfate solution for use in potable water treatment must meet all AWWA standards and be NSF/ANSI Standard 60 certified. Due to the economics of the existing processes, current ferric sulfate manufacturers don't have the flexibility of producing different or lower quality products suitable for other applications that requires different quality standards, such as industrial wastewater treatment and sulfate-based hydrometallurgical processes.

The advantage of the present process is that the quality of liquid ferric sulfate can be controlled to suit the needs of users who don't require the same quality of the product for portable water treatment. In remote areas where transport of liquid ferric sulfate is too costly, a bioleaching or bacterial oxidation plant based on the present process provides a cost effective method to obtain liquid ferric sulfate.

While preferred embodiments have been described above and illustrated in the accompanying drawings, it will be evident to those skilled in the art that modifications may be made without departing from this disclosure. Such modifications are considered as possible variants comprised in the scope of the disclosure.

1. A process for manufacturing liquid ferric sulfate from an iron sulfide material provided from ore tailings or mine tailings, the process comprising the step of:
contacting an aqueous solution containing the iron sulfide material with a thermotolerant bacteria culture capable of promoting oxidation of the iron sulfide material for producing liquid ferric sulfate effected without a sulfuric acid addition.

2. The process of claim 1, wherein the iron sulfide material comprises one of: pyrite, pyrrhotite, troilite, greigite, magnetite, mackinawite, marcasite and any combination thereof.

3. The process of claim 1, wherein contacting the aqueous solution containing the iron sulfide material with the thermotolerant bacteria culture occurs in one of: an agitated tank, an agitated reactor, a non-mechanically agitated column, a non-mechanically agitated tank, a non-mechanically agitated reactor, a pipe reactor, a reactor, a batch reactor, a continue reactor, a stacked heap, a dump leaching, a heap leaching, non-stacked piles, stacked piles, an aerated reactor, a non-aerated reactor and any combination thereof.

4. The process of claim 1, wherein the thermotolerant bacteria culture includes an optimum growth temperature of 40 to 45° C.

5. The process of claim 1, wherein contacting the aqueous solution containing the iron sulfide material with the thermotolerant bacteria culture occurs at a temperature in the range from 25 to 55° C.

6. The process of claim 1, wherein the thermotolerant bacteria culture is capable of promoting oxidation of the iron sulfide material at a temperature in the range from 25 to 55° C.

7. The process of claim 1, wherein contacting the aqueous solution containing the iron sulfide material with the thermotolerant bacteria culture comprises solubilizing the thermotolerant bacteria culture in the aqueous solution.

8. The process of claim 1, further comprising the step of grounding or crushing the iron sulfide material prior to contacting the aqueous solution containing the iron sulfide material with the thermotolerant bacteria culture.

9. The process of claim 1, wherein contacting the aqueous solution containing the iron sulfide material with the thermotolerant bacteria culture follows a chemical reaction defined by:
4FeS₂+15O₂+2H₂O→2Fe₂(SO₄)₃+2H₂SO₄ (i)

10. The process of claim 9, wherein the chemical reaction includes individual reactions defined by:
2FeS₂+7.5O₂+H₂O→2FeSO₄+2H₂SO₄ (1)
4FeSO₄+O₂+2H₂SO₄→2Fe₂(SO₄)₃+2H₂O (2)
FeS₂+Fe₂(SO₄)₃→3FeSO₄+2S (3)
2S+3O₂+2H₂O→2H₂SO₄ (4)

11. The process of claim 1, wherein contacting the aqueous solution containing the iron sulfide material with the thermotolerant bacteria culture occurs at a pressure in the range from 10 psi to 20 psi.

12. The process of claim 1, wherein contacting the aqueous solution containing the iron sulfide material with the thermotolerant bacteria culture occurs at a pressure of 14.7 psi (1 atm).

13. The process of claim 1, wherein the iron sulfide material comprise an iron sulfide material concentrate.

14. In a process for producing liquid ferric sulfate, the improvement consisting of:
contacting an aqueous solution containing an iron sulfide material with a thermotolerant bacteria culture capable of promoting oxidation of the iron sulfide material for producing liquid ferric sulfate effected without a sulfuric acid addition.
